(19) United States
(12) Patent Application Publication
(10) Pub. No.: US 2021/0140946 A1
(43) Pub. Date: May 13, 2021

(54) SYNCYTIOTROPHOBLAST EXTRACELLULAR VESICLES AS BIOMARKER FOR GESTATIONAL DIABETES MELLITUS

(71) Applicant: Oxford University Innovation Limited, Oxford (GB)

(72) Inventors: Manu VATISH, Oxford (GB); Ian SARGENT, Oxford (GB); Neva KANDZIJA, Oxford (GB); Wei ZHANG, Oxford (GB); Dionne TANNETTA, Oxford (GB)

(21) Appl. No.: 16/616,517

(22) PCT Filed: Jun. 1, 2018
(86) PCT No.: PCT/GB2018/051500

§ 371 Date: Nov. 25, 2019

(30) Foreign Application Priority Data
Jun. 1, 2017 (GB) 1708741.2

(51) Int. Cl. G01N 33/50; G01N 33/68

(52) U.S. Cl. CPC G01N 33/5076; G01N 33/689; G01N 2800/368; G01N 2333/96425; G01N 2800/50; G01N 2333/72; G01N 2800/042

(57) ABSTRACT

The present invention relates to a method of determining the gestational diabetic status of a pregnant subject, comprising providing a biological sample obtained from the subject; and determining the presence, and/or level, of syncytiotrophoblast extracellular vesicles, and/or insulin receptor, and/or DPPIV in the biological sample.

The present invention relates to novel biomarkers for gestational diabetes, and to uses of the novel biomarkers.

Pregnancy induces a series of intricate changes to the maternal metabolism, most notably progressive insulin resistance, which increases with advancing gestation. A 50%-60% increase in insulin resistance is apparent at 36 weeks gestation. Despite this, women are able to maintain euglycaemia due to commensurate increases in pancreatic islet β cell production of insulin. This paradigm constitutes the hallmarks of type II diabetes mellitus; hyperinsulinaemia in the presence of euglycaemia due to insulin resistance. In cases where the maternal ability to produce insulin is insufficient to meet the metabolic requirements of pregnancy, hyperglycaemia develops. There is therefore, a relationship between insulin resistance and insulin availability that is finely balanced in pregnancy.

The mechanisms that underlie gestational insulin resistance and hence gestational diabetes remain poorly understood. However, it is apparent that the insulin resistance associated with normal pregnancy is rapidly attenuated upon delivery of the placenta. Moreover, the placenta has been shown to release a number of factors that have been implicated in regulating insulin resistance (Barbour et al., (2007) Diabetes Care. https://doi.org/10.2337/dc07-s202). To date, only the expression of TNFα, a proinflammatory adipokine, has been shown to correlate with insulin resistance during pregnancy). While TNFα has been reported to downregulate insulin receptor signaling in vitro in numerous cell types, anti-TNFα therapy for the treatment of insulin resistance and diabetes in humans has been unsuccessful, suggesting that more complex mechanisms for signaling between the feto-placental unit and mother are involved.

If gestational diabetes is not detected and treated, it can increase the risk of serious birth complications for both mother and baby, including macrosomia, shoulder dystocia, premature birth, miscarriage and stillbirth. In addition to the increased risk of complications associated with gestation and delivery, there are also serious post-natal complications associated with gestational diabetes. For example, there is an increased risk that both mother and baby will develop type 2 diabetes later in life.

Currently, there is no method of determining whether a pregnant woman is likely to develop gestational diabetes. The only diagnostic test is an oral glucose tolerance test (OGTT), which is done late on in gestation, at around 24-28 weeks, and is dependent upon the patient having the disease. Moreover, the OGTT is not a particularly effective test, in part because of lack of compliance by patients who are required to fast before having the test. Fasting is also not healthy for the foetus. Accordingly, there is a need for new and improved methods for predicting the onset of and/or diagnosing gestational diabetes. The present invention fulfills these needs and further provides other related advantages.

Accordingly, in a first aspect of the invention there is provided a method of determining the gestational diabetic status of a pregnant subject, comprising:
(a) providing a biological sample obtained from the subject; and
(b) determining the presence, and/or level, of syncytiotrophoblast extracellular vesicles in the biological sample.

Syncytiotrophoblast extracellular vesicles are membrane bound vesicles that are constitutively shed from the syncytiotrophoblast and serve as placental derived signaling molecules which are deported into the maternal circulation. They comprise two modalities: microvesicles which are about 100 nm to about 1000 nm in size and exosomes which are about 50 nm to about 200 nm in size (Dragovic et al., (2015). Methods, 87, 64-74. https://doi.org/10.1016/j.ymeth.2015.03.028). In an alternative embodiment microvesicles are about 100 nm to about 1000 nm in size and exosomes are about 50 nm to about 100 nm in size. In a further embodiment microvesicles are about 200 nm to about 1000 nm in size and exosomes are about 50 nm to about 200 nm in size.

The method of the invention may comprise determining the level of syncytiotrophoblast microvesicles, syncytiotrophoblast exosomes, or a combination thereof, in a sample from a subject.

The syncytiotrophoblast extracellular vesicles may express an insulin receptor. The invention may use the syncytiotrophoblast extracellular vesicles to detect the level of insulin receptor in a sample.

The syncytiotrophoblast extracellular vesicles may express dipeptidyl peptidase 4 (DPPIV). DPPIV may be bound to the surface of syncytiotrophoblast extracellular vesicles. The invention may use syncytiotrophoblast extracellular vesicles to detect the amount of DPPIV in a sample.

In an embodiment, the level of either or both the insulin receptor and DPPIV in a sample may be used to determine the level of syncytiotrophoblast extracellular vesicles in a sample.

In another aspect, the invention provides a method of determining the gestational diabetic status of a pregnant subject, comprising:
a) providing a biological sample obtained from the subject; and
b) determining the level insulin receptor and/or DPPIV in the biological sample.

The number of syncytiotrophoblast extracellular vesicles may increase with the onset of gestational diabetes. The number of syncytiotrophoblast microvesicles may increase with the onset of gestational diabetes. The number of syncytiotrophoblast exosomes may increase with the onset of gestational diabetes. A greater number of syncytiotrophoblast microvesicles compared to syncytiotrophoblast exosomes may be indicative/diagnostic/predictive of gestational diabetes. Alternatively an increase in the number of both or either of syncytiotrophoblast microvesicles and syncytiotrophoblast exosomes may be indicative/diagnostic/predictive of gestational diabetes.

The level of insulin receptor and/or DPPIV may increase with the onset of gestational diabetes.

The term ‘gestational diabetic status’ includes any distinguishable manifestation of gestational diabetes. For example and without limitation, the presence or absence of gestational diabetes (diagnostic), the risk of developing gestational diabetes (predictive) or the stage of gestational diabetes.

The method of the invention may also include the step of measuring the level of circulating insulin in a subject.

The method of the invention may be used, for example, for any one of the following: to diagnose gestational diabetes; to assess the chance of a subject developing gestational diabetes, that is, to predict whether a subject is likely to develop gestational diabetes; and to advise on the prognosis of a subject with gestational diabetes.

Preferably, the method allows the diagnosis of gestational diabetes in a pregnant subject from the analysis of the level of syncytiotrophoblast extracellular vesicles in a sample provided by the subject. Alternatively, or additionally, the method may allow the diagnosis of gestational diabetes in a pregnant subject from the analysis of insulin receptor and/or DPPIV in a sample provided by the subject. Alternatively, or additionally, the method may allow the diagnosis of gestational diabetes in a pregnant subject from the analysis of insulin receptor and/or DPPIV and the level of syncytiotrophoblast extracellular vesicles in a sample provided by the subject.

The method may also allow the prediction of whether or not a pregnant subject is likely to develop gestational diabetes from the analysis of the level of syncytiotrophoblast extracellular vesicles in a sample provided by the subject, or from the analysis of the level of insulin receptor and/or DPPIV in a sample provided by the subject, or from a combination of both.

The term ‘level’ with respect to syncytiotrophoblast extracellular vesicles as used herein refers to the amount or concentration of syncytiotrophoblast extracellular vesicles contained in a biological sample.

The level of syncytiotrophoblast extracellular vesicles may be compared to the level in a control sample to allow for any inaccuracy or background in the test method used.

The method of the invention may further comprise the step of comparing the level of syncytiotrophoblast extracellular vesicles determined in (b) with one or more reference values. Alternatively, or additionally, the method of the invention may further comprise the step of comparing the level of insulin receptor and/or DPPIV determined in a sample with one or more reference values.

The reference value may be the level of syncytiotrophoblast extracellular vesicles, or the level of insulin receptor and/or DPPIV, in a sample obtained from a pregnant subject who does not have/does not go on to develop gestational diabetes. Preferably the reference and sample tested are compared at the same time during a pregnancy, for example both relate to levels observed between about week 19 and about week 21 of pregnancy. Additionally or alternatively, the reference value may be a previous level of syncytiotrophoblast extracellular vesicles, or the level of insulin receptor and/or DPPIV, observed in the subject at an earlier time period, for example a value obtained from a sample taken about 10, 9, 8, 7, 6, 5, 4, 3, 2, or 1 week earlier.

The skilled person will appreciate that by studying a large cohort of pregnant subjects and monitoring those which go on to develop gestational diabetes, it will be possible to define a level of syncytiotrophoblast extracellular vesicles, or a level of insulin receptor and/or DPPIV, above which it can be concluded a subject has or is highly likely to develop gestational diabetes.

In one embodiment, an increase in the concentration of syncytiotrophoblast extracellular vesicles, or the level of insulin receptor and/or DPPIV, in a sample compared to a reference value is indicative, diagnostic or predictive of gestational diabetes. For example, where the sample value is at least about 2-fold, about 3-fold, or about 4-fold more than the reference value this may be indicative, diagnostic or predictive of gestational diabetes. Additionally or alternatively, an increase of about 20% or more, about 30% or more, about 40% or more, about 50% or more, or about 60% or more may be indicative, diagnostic or predictive of gestational diabetes.

In an embodiment, if a sample of peripheral plasma obtained between 17 and 40 weeks into a subjects pregnancy contains greater than about 500 events (syncytiotrophoblast extracellular vesicles) per ml of plasma which are PLAP positive and DPPIV positive then the subject may be diagnosed as having, or being at high risk of developing gestational diabetes and should be treated accordingly.

The level of syncytiotrophoblast extracellular vesicles, and in particular the level of syncytiotrophoblast extracellular vesicles expressing insulin receptor and/or DPPIV, may be evaluated by any suitable method. For example, if vesicle/receptor levels are to be determined any of the group comprising immunoassays, spectrometry, western blot, ELISA, immunoprecipitation, slot or dot blot assay, isoelectric focusing, SDS-PAGE, antibody microarray immunohistological staining, radio immune assay (MA), fluoroimmunoassay, an immunoassay using an avidin-biotin or streptoavidin-biotin system, etc. and combinations thereof may be used. Other methods may also be used. These methods are well known to the person skilled in the art.

The term ‘biological sample’ defined herein refers to a sample of biological fluid obtained for the purpose of diagnosis or evaluation of a subject of interest. Preferred biological samples include, but are not limited to, blood, serum, and plasma. In addition, the person skilled in the art would realise that some test samples would be more readily analysed following a fractionation or purification procedure, for example, separation of whole blood into serum or plasma components.

The step of obtaining the sample preferably does not form part of the invention.

The method of the invention may be used in conjunction with an assessment of clinical symptoms to provide a more effective diagnosis of gestational diabetes.

According to another aspect of the invention, there is provided a method of treating gestational diabetes in a pregnant subject, comprising:
(a) obtaining a biological sample from the subject;
(b) determining the level of syncytiotrophoblast extracellular vesicles, and/or the level of insulin receptor and/or DPPIV, in the biological sample; and
(c) administering anti-diabetic therapy to the subject if the level of syncytiotrophoblast extracellular vesicles, and/or the level of insulin receptor and/or DPPIV, in the sample is indicative and/or diagnostic and/or predictive of gestational diabetes.

The anti-diabetic therapy may include, but is not limited to, any of insulin, metformin, an insulin receptor blocker, dialysis to remove syncytiotrophoblast vesicles, plasmapheresis, a DDPIV inhibitor, a gliptin or a combination thereof.

Where an increase in DPPIV is observed the subject may be administered a DDPIV inhibitor, such as a gliptin.

According to another aspect of the invention, there is provided a kit for use in determining the gestational status of a pregnant subject comprising at least one agent for determining the level of syncytiotrophoblast extracellular vesicles, and/or the level of insulin receptor and/or DPPIV, in a biological sample obtained from the subject.

The agent may be an antibody.

The kit may further comprise instructions suitable for operational parameters in the form of a label or separate insert. The instructions may inform a consumer about how to collect the sample.

The kit may also comprise one or more syncytiotrophoblast extracellular vesicle samples to be used as standards for calibration and comparison. The kit may comprise instructions to compare the level of syncytiotrophoblast extracellular vesicles detected in a sample with a calibration sample or chart. The kit may also include instructions indicating what level of syncytiotrophoblast extracellular vesicles is diagnostic and/or predictive of gestational diabetes.

According to a further aspect, the invention provides the use of the determination of the level of syncytiotrophoblast extracellular vesicles, and/or the level of insulin receptor and/or DPPIV, in a biological sample as a means of assessing the gestational diabetic status in a pregnant subject.

According to a further aspect, the invention provides receiving identification that a pregnant subject is suffering from gestational diabetes, wherein identification is provided by determining the level of syncytiotrophoblast extracellular vesicles, and/or the level of insulin receptor and/or DPPIV, in a blood sample, and treating the subject by administering an anti-diabetic therapy.

The invention may further provide a method of determining the risk of a woman developing Type 2 diabetes post-partum, the method comprising the determining the level of insulin receptor and/or DPPIV positive syncytiotrophoblast extracellular vesicles in a blood sample obtained from a mother at delivery.

Syncytiotrophoblast extracellular vesicles may be referred to herein as STB-EVs or STBEVs, syncytiotrophoblast extracellular microvesicles may be referred to STB-MVs or STBMVs and syncytiotrophoblast extracellular exosomes may be referred to as STB-EXs or STBEXs.

The skilled person will appreciate that preferred features of any one embodiment and/or aspect of the invention may be applied to all other embodiments and/or aspects of the invention.

There now follows by way of example only a detailed description of the present invention with reference to the accompanying drawings, in which:

FIG. 1 shows expression of insulin receptor (IR) in placental tissue and syncytiotrophoblast extracellular vesicles (STB-EV). FIG. 1A shows immunoblot analysis demonstrating the expression of IR in placental lysate and STB-EV preparations comprising microvesicles (150 KP) and exosome (10 KP) preparations (20 μprotein/lane). FIG. 1B shows the concentrations of IR in placental lysates and STB-EV determined by ELISA. Bars=mean±standard error of the mean, **=p<0.01. FIG. 1C shows immuno-histochemistry data demonstrating the expression of IR in placental tissue at 40× magnification (aiii) and 20× magnification (ai). The corresponding control sections are presented in (aii) and (aiiii) at 40× and 20× magnifications respectively (representative images, n=5).

FIG. 2 uses magnetic immunobead depletion experiments to show the level of expression of PLAP and the insulin receptor (IR) on STB-MVs and STB-EXs and demonstrates that PLAP is co-expressed with the IR on these vesicles. PLAP expression demonstrates the placental origin of the STB-EVs. More specifically, FIG. 2 shows representative immunoblot images of IR and PLAP co-expression in the microvesicles and exosomes pool showing untreated vesicles, and vesicles precipitated by anti-IR coated dynabeads or anti-PLAP coated dynabeads.

FIG. 3 shows STB-MV dependent depletion of insulin from plasma. The data shows the mean proportions of insulin remaining in three human plasma samples following incubation with three STB-MV preparations. STB-MV preparations (n=3) were added at concentrations of 0, 10, 100, 200 and 500 μg/ml to three platelet poor plasma preparations (n=3) from non-pregnant volunteers and incubated for 30 minutes at room temperature. The plasma was centrifuged at 30,000 g at 4° C. for 1 hour to pellet the STB-MV. The supernatant was collected and analysed using an insulin ELISA (R&D Systems, UK, DINS00) as described by the manufacturer.

FIG. 4 shows the flow cytometry gating strategy for detecting the co-expression of PLAP and IR on placental perfusion or chorionic villous explants derived STB-MVs. Flow cytometric analysis of STB-MVs stained with anti-PLAP antibody alone (FIG. 4a), or both anti-PLAP and anti-IR antibodies (n=6) (FIG. 4b) is shown. FIG. 4c shows the percentage insulin receptor positivity of six STB-MV isolated from chorionic villous explants determined by flow cytometry analysis (percentage of total STB-MV).

FIG. 5 shows representative plots in which flow cytometry has been used to look at PLAP and IR expression in 100 l of platelet poor plasma from the uterine vein or the peripheral vein from 3 subjects (PPP-1, -2 and -3). The blood sample were taken at birth. The data shows that there are more “events” in the uterine blood than in the peripheral blood, demonstrating the STB-EVs are derived from the placenta, but also demonstrates the STB-EVs can be detected in the peripheral blood.

FIG. 6 shows graphically the data depicted in FIG. 5, and confirms the contribution of the placenta to the circulating levels of insulin receptor (IR) during pregnancy.

FIG. 7 compares the STB-EVs in peripheral plasma at birth/term in a control subject (who does not have gestational diabetes) with those in a subject with gestational diabetes (GDM). The results show an increase in the overall number of STB-EVs, and an increase in PLAP positive and IR positive STB-EVs in mothers with GDM. The number of PLAP positive and IR positive STB-EVs was determined using flow cytometry.

FIG. 8 demonstrates that DPPIV is expressed on the placenta surface. Paraffin fixed 10 μm normal placental tissue sections were incubated for 24 hours with 1:2000 anti-DPPIV (ab28340, AB cam) antibodies before being stained with hematoxylin. The results are shown in the micrographs in FIG. 8, which clearly show expression of DPPIV on the placental surface. The scale bar is 100 μm.

FIG. 9 demonstrates that PLAP and DPPIV are expressed on the surface of the placenta. The immunoblot in FIG. 9 clearly shows that both PLAP and DPPIV are expressed on the placenta surface and on isolated STB-MVs and STB-EXs.

FIG. 10 shows the percentage of co-expression of DPPIV and PLAP on placental perfusion derived STB-MVs using flow cytometry. Flow cytometric analysis of STB-MVs stained with anti-PLAP antibody alone, or both anti-PLAP and anti-DPPIV antibodies (n=3) are shown.

FIG. 11 shows the results of Dynabead depletion experiments to demonstrate that DPPIV and PLAP are co-expressed on STB-EXs. Dynabeads carrying anti-DPPIV antibodies were incubated overnight with a preparation of STB-EXs. The beads were then removed magnetically and the bound vesicles were analyzed using western blotting. Analysis of the particle concentration and size distribution profile of STB-EXs were carried out using Nano Sight Tracking Analysis (NTA). STB-EXs prior (solid line) and supernatant from post incubation with a) anti-DPPIV Dynabeads (dotted line) and anti-PLAP (dashed line) or b) anti-IgG1 Dynabeads (dotted line) and anti-IgG2a Dynabeads (dashed line) are shown on FIG. 11.

FIG. 12 shows the results of Dynabead depletion experiments to demonstrate that DPPIV and PLAP are co-expressed on STB-MVs. Dynabeads carrying anti-DPPIV antibodies were incubated overnight with a preparation of STB-MVs. The beads were then removed magnetically and the bound vesicles were analyzed using western blotting. Analysis of the particle concentration and size distribution profile of STB-MVs were carried out using NTA. STB-MVs prior (solid line) and supernatant from post incubation with a) anti-DPPIV Dynabeads (dotted line) and anti-PLAP (dashed line) or b) anti-IgG1 Dynabeads (dotted line) and anti-IgG2a Dynabeads (dashed line) are shown on FIG. 12.

FIG. 13 demonstrates the potential of DPPIV as a pharmacological target for the treatment of gestational diabetes. More specifically, FIG. 13 shows that the activity of DPPIV can be inhibited by the addition of the gliptin Vildagliptin™—a drug used to treat Diabetes Type 2. 10 μg/mL of STB-MVs and STB-EXs pre-incubated for 15 min with different concentrations of Vildagliptin, DPPIV specific inhibitor, showed reduced DPPIV activity compared to control.

FIG. 14 demonstrates an increase in DPPIV activity in exosomes recovered from the post birth placenta of a woman with GDM compared to the exosomes recovered from the post birth placenta of a woman without GDM. The data clearly demonstrates that DPPIV activity may be used to diagnose, monitor or predict GDM in a pregnant subject.

FIG. 15 compares plasma derived from the uterine vein and the peripheral vein of subjects post birth. The level of PLAP positive event and PLAP and DPPIV positive events in each sample is compared. The results show that higher levels of positive events are observed in the uterine vein samples demonstrating that the STB-EVs are derived from the placenta. Furthermore, the results show that the STB-EVs can be detected in plasma derived from the peripheral blood, and thus peripheral blood may be used as sample material when screening pregnant women for gestation diabetes.

FIG. 16 shows in graphical form the results shown in FIG. 15.

FIG. 17 compares the levels of PLAP positive/DPPIV positive STB-EVs and PLAP only positive STB-EVS in plasma derived from peripheral blood samples from control subjects (who have just had a baby and did not have GDM) and from test subjects (who have just had a baby and did have GDM). The results presented clearly show elevated levels of PLAP positive/DPPIV positive STB-EVs in the peripheral plasma from mothers with GDM. Form this data it is predicted that if a mother has greater than 2000 STB-EVs/ml in their peripheral plasma at term then the mother had GDM. If a level of about 500 is observed at about 20 weeks pregnancy this is likely to be predictive that the mother will get, or already has, GDM.

### SYNCYTIOTROPHOBLAST EXTRACELLULAR VESICLES AND INSULIN RECEPTORS

Materials and Methods

Patient Information

Blood samples from pregnant patients and placentas were obtained from women undergoing elective caesarean section at 37-40 weeks at the John Radcliffe hospital, Oxford. Blood samples from non-pregnant patients were obtained from healthy volunteers with no previous pregnancies or known metabolic dysfunctions.

STB-EV Isolation and Purification

STB-EVs were isolated from placentas using the dual lobe perfusion model previously described (Dragovic et al., (2015). Methods, 87, 64-74. https://doi.org/10.1016/j.ymeth.2015. 03. 028). Briefly, placentas were obtained from women immediately after caesarean section and a single lobe was perfused for three hours. Perfusate from the maternal interface of the placenta was processed using a series of filtration and ultracentrifugation steps as described by Dragovic et al., 2015 to allow for fractionation of the exosome and microvesicle populations. Five colour flow cytometric analysis was used to verify the microvesicle fractions were of a high purity using the antibodies and procedure described in Dragovic et al., 2015. STB-EVs were stored at −80° C. Nanoparticle tracking analyses was used to confirm that the size and profile of the fractionated vesicles was consistent with exosomes and microvesicles.

Preparation of Platelet Poor Plasma

Blood was collected in 4.5 ml vacutainers containing 0.105M buffered sodium citrate (BD Biosciences) and processed immediately. Platelet poor plasma was generated by centrifuging whole blood at 1500 g for 15 minutes. The overlying plasma was centrifuged at 13000 g for two minutes to pellet the platelets. The supernatant was frozen at −80° C.

Detection of Insulin Receptor by Immunoblotting

Placental lysates were prepared from sections of fresh placentas taken from ˜1 cm beneath the decidua. Protein concentrations of STB-EV and placental lysates were determined using a BCA protein assay kit (Thermo Scientific, UK). Immunoblot assays were carried out as previously described (Collett et al (2012). PLoS ONE, 7(1). https://doi.org/10.1371/journal.pone.0030453). Primary antibodies used were anti-insulin receptor (R&D Systems, catalogue no. 15441) and an in house anti-placental alkaline phosphatase (NDOG2) (reference) antibody both used at concentrations of 1 μg/ml.

Detection of Insulin Receptor in STB-EV and placenta by ELISA

IR in placental lysates (n=6) and STB-EV preparations (n=6) were measured by ELISA (R&D Systems, catalogue number DYC1544) according to the manufacturer's instructions. Placental lysates and STB-EVs were assayed at concentrations of 0.05 mg/ml and 0.005 mg/ml respectively to obtain readings within the assay range.

Localisation of Insulin Receptor in Placental Sections determined by Immunohistochemisty

Immunohistochemistry was carried out on paraffin embedded placental tissue sections (n=5). Antigen retrieval was performed by immersing sections in 10 mM, pH 6 sodium citrate buffer for 3 minutes at 125° C. in a pressure cooker. Endogenous peroxidases were quenched by incubating sections in 0.3% hydrogen peroxide for 15 minutes. Sections were blocked for an hour with 10% PBS-fetal calf serum (FCS). Sections were incubated overnight at 4° C. in 1:10 dilution anti-insulin receptor antibody (Atlas antibodies, catalogue number HPA036302) diluted in 1% PBS-FCS (experimental) or 10% PBS-FCS alone (control). Sections were incubated with EnVision Flex Rabbit Linker (Dako, UK, SM805) for signal amplification and EnVision Flex/HRP (Dako, UK, SM802) secondary antibody. Sections were incubated with DAB substrate and counterstained with haematoxylin.

Flow Cytometry—PLAP and IR Staining of STB-EV Isolated by Placental Perfusion

STB-MV preparations (n=6) were assessed for PLAP and IR expression using two colour flow cytometry. Prior to use PBS, Fc receptor block and antibodies were filtered through a 0.2 μm filter, the flow cytometer flow rate was set to 11-12 μl/min as determined by TruCount beads (manufacturer) and the background event rate was <1000 events/minute. The volume of STB-MV used per test from individual samples was defined as the volume of STB-MV which, in a volume of 300 μl of PBS, gave an event rate of ˜250 events/second. STB-MV samples were blocked with 10 μl of Fc receptor at 4° C. for ten minutes before being stained with antibodies for 15 minutes at room temperature in a staining volume of 100 μl. STB-MV were stained with anti-IR-APC, PLAP-PE and the corresponding controls (see Table 1). STB-MV were topped up with PBS to 300 μl and analysed on a flow cytometer (Becton Dickinson LSR II). 20,000 events were collected for each test. The negative gates for staining were determined using fluorochrome minus one (FMO) tests and set at 1%. Data was analysed using Diva flow cytometry (Becton Dickinson) and FlowJo (FlowJo LLC) software.

TABLE 1
Antibodies used for analysis of STB-MV isolated by placental perfusion and detected in plasma.

| | | | Final | | |
|---|---|---|---|---|---|
| | Clone | Negative Control | Concentration | | Specificity |
| Fluoescent Label | | | | | |
| Bio-Maleimide (BIODIPY FL N-(2- | N/A | N/A | 0.25 | μg/ml | Thiol reactive dye- |
| aminoethyl) maleimide)-FITC | | | | | general cell marker |
| Antibodies | | | | | |
| CD235a-PEVio770^{a} | | REA control PEVio770^{a} | | | Erythrocytes and |
| Cat no. 130-100-258 | | Cat no. 130-104-616 | | | erythroid precursor |
| | | | | | cells |
| HLA Class II DR DP DQ-PEVio770a | | REA control PEVio770^{a} | | | Maternal nucleated cells |
| Cat no. 130-104-828 | | Cat no. 130-104-616 | | | |
| NDOG2-PE^{b} | N/A | IgG1-PE^{b} | 0.5 | μ/ml | Syncytiotrophoblast |

| | | |
|---|---|---|
| IR-APC^{a} | REA control APC^{a} | Insulin Receptor |
| Cat no. 130-103-653 | Cat no. 130-104-614 | |
| CD41 (used in the second set | | |

^{a}Miltenyi Biotec Ltd. UK
^{b}Biolegend Ltd. UK

Insulin ELISA—Plasma Depletion Assay

STB-MV preparations (n=3) were added at concentrations of 0, 10, 100, 200 and 500 μg/ml to three platelet poor plasma preparations (n=3) from non-pregnant volunteers and incubated for 30 minutes at room temperature. The plasma was centrifuged at 30,000 g at 4° C. for 1 hour to pellet the STB-MV. The supernatant was collected and analysed using an insulin ELISA (R&D Systems, UK, DINS00) as described by the manufacturer.

Flow Cytometry—Detection of STB-MV Isolated From Plasma

Four colour flow cytometer was used to identify PLAP and IR positive particles from plasma samples. Flow cytometer was set up as described above. 100 μL of platelet-poor plasma was labelled with anti-Plap-PE, anti-IR-APC and potential contaminating markers (anti-CD41a-PE-Cy7 as a marker for platelet EVs, anti-CD235a-PE-Cy7 as a marker for red blood cells' EVs, anti-HLAClass I-PE-Cy7 and anti-HLA Class II-PE-Cy7 as markers for all the EVs except from those derived from STB or red blood cells). After 15 minutes incubation at 4° C., stained plasma was filtered using Ultrafree-MC/Durapore-PVDF centrifugal filters (2 minutes, 800 g). EVs were recovered from the top of the filter unit with 100 μL of filtered PBS following with Bio-Maleimide-FITC staining. Filtrate was used to determine Bio-Maleimide positive and contaminating markers negative gate. Events that fell into this gate were then further analysed for PLAP and IR binding. In order to determine negative gates for anti-IR-APC and anti-PLAP-PE staining, detergent (Nonidet P-40, New England Biolabs, UK) treated samples were used and set at 1%. Prior to data acquisition, samples were toped up with filtered PBS to 500 μL. Each sample was run for 10 minutes. Both data analysis and figures generation were carried out using FlowJo version 10.1 (Tree Star Inc, Ashland, US).

Results

Expression of Insulin Receptor in Placenta and STB-EV

Expression of insulin receptor (IR) was verified in 12 placental lysates and 12 STB-EV preparations (6×STB-exosome (STB-EX) and 6×STB-microvesicle (STB-MV) sub-fractions) isolated from term placentas by western blot. Immunoblot analysis of microvesicles and exosomes derived from the same placentas showed no significant differences in insulin receptor expression (FIG. 1A). The degree of insulin receptor expression in placenta and STB-EV was quantitated by ELISA (FIG. 1B). Mean insulin receptor concentrations were 90.96±26.2 and 1548±438 (ng/mg) (mean±STDEV) in placenta and STB-EV respectively; demonstrating that IR expression is significantly greater in STB-EV than in placental lysates (p=0.005). The presence of insulin receptor in the placenta was further confirmed by immunohistochemistry (FIG. 1C) which demonstrated insulin receptor expression across the placental villi with an enrichment in the trophoblast layers. Insulin receptor was present in both cytotrophoblast and syncytiotrophoblast layers and in some areas was further enriched at the syncytiotrophoblast apical membrane from which STB-EV are shed. In contrast, staining for IR was absent in controls. From herein, analysis was carried out on STB-MV but not STB-EX as STB-EX are smaller than the lower limit of detection of flow cytometry. Moreover, previous work has shown that STB-MV are highly enriched for PLAP with minimal contamination of endothelial cell, red blood cell and platelet derived microvesicles (Tannetta et al (2013) PLoS ONE, 8(2). https://doi.org/10.1371/journal.pone.0056754).

Co-expression of IR and PLAP on STB-EV

It has previously been shown that placental alkaline phosphatase (PLAP) serves as a placental specific marker in flow cytometry. Six STB-MV preparations were analysed by three colour flow cytometry to assess PLAP and IR expression (FIG. 4A-C). All STB-MV preparations showed greater than 90% PLAP positivity confirming the placental origin of the vesicles and high levels of sample purity. A sub-population of IR+PLAP+STB-MV was present in each sample (mean 29.6%±STDEV) (FIG. 4B-C), indicating the co-expression of PLAP and IR. No vesicles were seen to be IR positive and PLAP negative.

Co-expression of PLAP and IR on STB-MVs was further confirmed by magnetic bead immunoprecipitation. The untreated STB-MV pool (control) and bead precipitates (precipitated by anti-PLAP and anti-IgG1 antibody coated beads) were analysed by immunoblotting (FIG. 2) for PLAP and IR expression. The protein extracted with anti-PLAP antibody coated beads showed a PLAP band of comparable size to that from the untreated STB-MV pool, indicating the beads had successfully precipitated PLAP from the STB-MV sample. The anti-PLAP bead precipitate also showed IR expression, further indicating co-expression of PLAP and IR on STB-MV moieties. The size of the IR band observed in the anti-PLAP bead precipitate was comparable to that from the untreated STB-MV, suggesting that all STB-MV which express IR, also express PLAP. IgG1 antibody coated beads led to a negligible degree of PLAP precipitation indicating specific binding of the PLAP antibody. The absence of an IR band in the IgG1 precipitate suggests that PLAP and IR are specifically linked on microvesicles.

STB-EV Mediated Depletion of Insulin from Plasma.

Having detected the expression of IR on STB-EVs, the ability of STB-MVs to bind and deplete insulin from plasma samples was investigated. Samples were incubated with 0-500 μg/ml STB-MV, ultracentrifuged to allow removal of the STB-MVs, and assessed for insulin receptor concentration by ELISA (FIG. 3). Insulin concentration was conversely associated with STB-MV dose with 55% of insulin being depleted at the maximum STB-MV dose of 500 μg/ml. This dose dependent depletion of insulin from plasma mediated by STB-MVs indicates that STB-MVs are functionally active and have a high affinity for insulin binding.

STBEVs in Uterine Vs Peripheral Plasma

Four colour flow cytometry was used to investigate the circulating levels of placental derived EVs in uterine and peripheral plasma (n=6). Syncytiotrophoblast microvesicles carrying IR are present in significantly higher amounts in the uterine vein (IR/PLAP double positive events per mL of plasma) compared with matched peripheral vein plasma (p<0.05), confirming the contribution of the placenta to the circulating levels of IR during pregnancy (FIG. 5, FIG. 6). The number of PLAP positive events was also significantly higher in uterine vein compared to peripheral plasma, again confirming placental origin of circulating particles.

STBEVs in GDM Vs Normal Plasma

In this study for the first time, placental derived EVs carrying IR are identified in peripheral plasma. The levels of those particles were investigated in the peripheral circulation of woman with normal and GDM pregnancies using flow cytometry. A higher co-expression of IR and PLAP on the STBEVs was observed in the GDM pregnancies compared to normal pregnancy (FIG. 7).

Discussion

The data presented herein demonstrates a role for STB-EVs as regulators of insulin availability and drivers of gestational insulin resistance. Furthermore the use of STB-EVs and/or the insulin receptor as markers for diagnosing or predicting gestational diabetes is taught.

It is demonstrated by western blot and flow cytometry that a subset of STB-EVs, both isolated by placental perfusion and chorionic villous explant culture, express insulin receptor. Moreover, it is demonstrated by immunoprecipitation and flow cytometry that STB-MVs moieties which express IR also co-express the placental specific marker PLAP.

Importantly, it is demonstrated that IR+STB-MV released from the placenta are functionally active as they are able to consistently deplete significant proportions of insulin from plasma in a dose dependent manner with 55% of insulin being depleted at the top STB-MV dose (100 μg/mL). Finally the presence of IR+PLAP+STB-MV in vivo in the peripheral and uterine vein plasma of pregnant women was identified. This is the first time functional soluble IR has been identified. The identification of circulatory IR+PLAP+STB-MV, but not IR+PLAP-STB-MV in plasma suggests that placental vesicles serve as the major cellular source of soluble IR.

Syncytiotrophoblast Extracellular Vesicles and Dipeptidyl Peptidase IV (DPPIV)

Materials and Methods

Tissue Samples

Normal and GDM placentas were obtained from consenting and fully informed volunteers during caesarean delivery. Whole blood was collected in citric acid tubes and within 30 minutes of collection tubes were centrifuged at 1 500 g for 15 minutes in order to separate cells from supernatant (platelet-poor plasma). Normal pregnancy included women who were normotensive, without proteinuria or GDM. GDM was defined for those patients who had a fasting plasma glucose level >5.6 mmol/L or >7.8 mmol/L two hours after a 75 g glucose load as part of the oral glucose tolerance test, performed at 26-28 weeks gestation. This study was approved by Oxfordshire research Ethics Committee C (H0604/148).

Immunohistochemistry

Placental sections (10 μm) were deparaffinsed in Histoclear (Sigma Aldrich, UK), and rehydrated in ethanol (Sigma Adrich, UK). For antigen retrieval slides were heated in 10 mM sodium citrate, pH 6 (Sigma UK) for ten minutes and cooled at room temperature.

Endogenous peroxidase activity was blocked with 3% H2O2 in PBS (Sigma Aldrich, UK) in order to prevent high background staining. The slides were rinsed with water prior to blocking for non-specific antibody binding using 10% Fetal Calf Serum (Sigma Aldrich, UK) in PBS-T (PBS with Tween 20, Sigma Aldrich, UK) at room temperature for one hour. The sections were then incubated overnight at 4° C. with 1% FCS and 0.5 μg/mL of anti-CD26 primary antibody (OriGene, US) in PBS-T. For the negative control, the primary antibody was replaced with non-immune mouse IgG1 (Biolegend, UK). The sections were washed in PBS and were incubated in humidifying chamber at room temperature for 1 hour with a anti-mouse IgG secondary antibody (Life Technologies, UK). After washing with 0.01% PBS-T (PBS with Tween 20, Sigma Aldrich, UK), the slides were stained with DAB (Vector Laboratories, US). All slides were washed in 0.01% PBS-T and in distilled water before the nuclei were counter-stained with Hematoxylin (Thermo Fisher, UK) for 10 minutes. The slides were then dehydrated in ethanol and Histoclear. Finally, the slides were covered with size No 1° cover slips (VWR International, UK) using Depex mounting medium (Sigma, UK), and a Leica DMIRE 2 microscope was used to view the sections while Hamamatsu Orca digital camera and HCl software were used to take images.

Isolation and Characterisation of STBEVs

STBEVs were obtained from a Dual Lobe Placental Perfusion system as previously described by us (REF). Briefly, the placentae were perfused for 3 hours and the maternal side perfusate collected (mPerf). Fresh mPerf was centrifuged (Beckman Coulter Avanti J-20XP centrifuge and Beckmen Coulter JS-5.3 swing out rotor) twice at 1,500 g for 10 minutes at 4° C. to remove erythrocytes and large cellular debris. The supernatant was collected and spun at 10,000 g (Beckman L80 ultracentrifuge and Sorvall TST28.39 swing out rotor) for 35 minutes at 4° C. to pellet ‘large’ microvesicles (300 nm-1 μm in diameter). The resultant pellet (10 KP) was resuspended in sterile PBS. The remaining supernatant was passed through a 0.2 μm stericup filter (Millipore), followed by spun at 150,000 g for 2 hours and 5 minutes at 4° C. (Beckman L80 ultracentrifuge and Sorvall TST28.39 swing out rotor) in order to pellet the exosomes (100 μm-300 μm). Pellets containing enriched exosomes (150 KP) were pooled and resuspended in sterile PBS. Both fresh pellets (10 KP and 150 KP) were assessed for protein concentration using BSA protein assay kit (Thermo Fisher, UK) and size characterization with Nanoparticle Tracking Analysis (Nanosight NS500, Malvern Instruments, UK) prior to subsequent analysis. Additionally, 10 KP was analysed for STBEVs marker PLAP using Flow Cytometer.

Nanoparticle Tracking Analysis

Measurements of particle diameter and concentration were conducted using NanoSight NS500 (Malvern Instruments, UK) equipped with sCMOS camera and nanoparticle Tracking Analysis software version 2.3, Build 0033 (Malvern, UK). The size distribution profiles and concentration of exosomes and microparticles were measured using the protocol described by us (REF 2015). Prior to data collection, samples were diluted with an appropriate amount of PBS, which was previously filtered through 200 nm Minisart filters. Each sample was measured five times at 25° C. and the mean value±standard deviation was calculated. Before the measurements of the samples were performed, the instrument was calibrated using silica 100 nm microspheres (Polysciences, Inc.).

Immunoblotting

Placental sections from the maternal side were homogenised in HEPES lysis buffer (Sigma, UK) to obtain placental lysate. 30 μg/well of placental lysates, microvesicles or exosomes (n=8) were denatured at 95° C. in Laemmli Sample Buffer (Bio-Rad, UK) and run on Mini-Protean TGX NuPAGE gel cassettes (Bio-Rad, UK) for 1 hour at 150 V. Proteins were transferred onto an Immuno-blot polyvinylidene difluoride membrane (Bio-Rad Laboratories, UK) in a Novex Semi-Dry Blotter (Life Technologies, UK) for 60 minutes at 25V. The membranes were then incubated for 1 hour at room temperature in 5% (w/v) Blotto (Alpha Diagnostic, UK) in TBS-T (Tris-buffered saline solution with 0.1% Tween-20) prior to overnight incubation at 4° C. with primary antibodies: 1 μg/mL pf DPPIV/CD26 antibody (R&D System, USA) or 1 μg/mL anti-PLAP (placental alkaline phosphatase) (NDOG-2, in house). Next morning membrane was washed in TBS-T followed by the incubation for 1 hour at room temperature with the corresponding horse radish peroxidase conjugated secondary antibody (Life Technologies, UK) in Blotto/0.1% TBS-T. Finally, the membranes were washed again in TBS-T and enhanced chemiluminescence substrate (ECL Western Blotting substrate, Thermo Scientific, UK) was used prior to exposure to Amersham Hyperfilm ECL (GE Healthcare, UK).

Magnetic Bead Depletion

Dynabeads M-280 Sheep Anti-Mouse IgG (Life Technologies, UK) conjugated to anti-CD26 antibody (Biolegend, USA) or anti-PLAP antibody (NDOG-2, in house) were prepared for the immunodepletion experiment according to the manufacturer's instructions. Dynabeads coated with anti-IgG1 antibody (Biolegend, USA) or anti-IgG2a antibody (Dako, UK) were used as control. Briefly, the superparamagnetic beads (50 μL) were resuspended in Washing Buffer (Ca²⁺ and Mg²⁺ free PBS with 0.1% BSA and 2 mM EDTA, pH 7.4), pelleted with a magnet, and resuspended in buffer again. 6 μg of anti-PLAP, anti-CD26, anti-IgG1 or anti-IgG2a antibody was respectively added to the beads, followed by the incubation on a rotating plate overnight at 4° C. The following morning, the beads were placed on the magnet and the supernatant was discarded in order to remove the excess antibody. After coupling the beads with antibody, 25 μg of 10 KP or 150 KP Pool (containing 4 individual samples from NP and incubated with 10 μL of anti-human Fc receptor blocking reagent for 10 mins at 4° C.) was added to them, followed by an overnight incubation at 4° C. Bound and unbound EVs were separated using a magnetic separator (Dynal, Norway), and the pellets containing bound Ab-positive STBEVs were analysed by Immunoblotting. Supernatants, with Ab-negative STBEVs, were analysed by NTA and used to calculate the percentage of STBEVs bound to beads as:

$$\% DPPIV or PLAP depleted particles = \frac{[ Total ] - [ DPPIV or PLAP negative ]}{[ Total ]} * 100$$

Flow Cytometric Analysis

10 KP Analysis

10 KP was analysed using BD LSRII flow cytometer (BD Biosciences). Flow cytometry setup was carried out using CS&T instrument setup beads (BD Bioscience). TruCount tubes were used to establish Flow rate (500 μL of filtered PBS was added to known number of fluorescent beads) and the background event rate was set up at <1000 events/minute. Appropriately diluted 10 KP (defined as the volume of 10 KP which, in a volume of 300 μl of PBS, gave an event rate of ˜300 events/second) was incubated with 10 μL of Fc receptor blocker (Miltenyi, UK) for 10 minutes at 4° C. After blocking, samples were labelled with anti-PLAP-PE, anti-DPPIV-APC and Biomaleimide-FITC as EV membrane marker (BODIPY N-(2-aminoethyl)-maleimide Thermo Fisher, UK) for 15 minutes at room temperature in a staining volume of 100 μL. Isotype controls were matched to their respective antibodies according to the concentration, fluorochrome type and heavy chain. Prior to data acquisition samples were topped up with PBS to 300 μL. For each sample 100,000 events were collected. The negative gates for staining were determined using isotype control tests and set at 1%, and both data analysis and figures generation were carried out using FlowJo version 10.1 (Tree Star Inc, Ashland, Oreg.).

Plasma Samples Analysis

Four colour flow cytometer were used to identify PLAP and DPPIV positive particles from plasma samples. Flow cytometer was set up as described above. 100 μL of platelet-poor plasma was labelled with anti-Plap-PE, anti-DPPIV-APC and potential contaminating markers (anti-CD41a-PE-Cy7 as a marker for platelet EVs, anti-CD235a-PE-Cy7 as a marker for red blood cells' EVs, anti-HLAClass I-PE-Cy7 and anti-HLA Class II-PE-Cy7 as markers for all the EVs except from those derived from STB or red blood cells). After 15 minutes incubation at 4° C., stained plasma was filtered using Ultrafree-MC/Durapore-PVDF centrifugal filters (2 minutes, 800 g). EVs were recovered from the top of the filter unit with 100 μL of filtered PBS following with Bio-Maleimide-FITC staining. Filtrate was used to determine Bio-Maleimide positive and contaminating markers negative gate. Events that fell into this gate were then further analysed for PLAP and DPPIV binding. In order to determine negative gates for anti-DPPIV-APC and anti-PLAP-PE staining, detergent (Nonidet P-40, New England Biolabs, UK) treated samples were used and set at 1%. Prior to data acquisition, samples were toped up with filtered PBS to 500 μL. Each sample was run for 10 minutes. Both data analysis and figures generation were carried out using FlowJo version 10.1 (Tree Star Inc, Ashland, US).

DPPIV Enzyme Activity Assay

DPPIV enzyme activity of the STBEVs was determined by use of a DPPIV-Glo Protease Assay (Promega, UK) as per the manufacturer's instructions. The DPPIV-Glo Reagent was added to 96 white well plates along with either sample, Tris-BSA as a blank or purified DPPIV enzyme in Tris-BSA as standard (Recombinant human CD26 protein, Abcam, UK), followed by incubation at room temperature for 30 minutes. Luminescence was measured using a FLUOstar Omega (BMG Labtech, UK) machine. Blanks are taken as a record of background luminescence and are subtracted from the results. Quantification was achieved by reference to calibration curve produced from recombinant human DPPIV protein standards at concentration ranging from to 1 ng/mL to 0.00625 ng/mL. Finally, both 10 KP and 150 KP were treated with DPPIV specific inhibitor—vildagliptin in order to measure the residual DPPIV activity. The residual DPPIV activity was calculated by comparing DPPIV enzymatic activity after the treatment with vildagliptin with non-treated samples.

Statistics

Statistical analyses were performed using the Prism 3.0 (GraphPad Software Inc, San Diego, Calif.). Analyses of means of two groups were performed using the independent two-sample Student's t-test, with group means considered significantly different when P<0.05 annotated in figures with (*).

Results

The Human Placenta and Placental Syncytiotrophoblast Extracellular Vesicles Express DPPIV

DPPIV was visualised on normal placental sections using a monoclonal anti-DPPIV antibody, showing enhanced localisation to the syncytiotrophoblast layer, whereas no staining was detectable when non-specific negative control antibody replaced the primary antibody (FIG. 8) (n=2).

Since the aim of this study was to investigate the expression and activity of DPPIV on STBEVs, extracellular vesicles isolated from placental perfusion were analysed by Western blotting. Western blots of both STB-MVs and STB-EXs (n=3) clearly demonstrated that DPPIV was present in both fractions (FIG. 9). Immunoblotting analysis of normal placental lysates (n=2) also showed the expression of DPPIV in the placental tissue. Recombinant human DPPIV protein was used as a positive control to confirm the immunoblot band observed at 110 kDa was DPPIV. Additionally, all samples were also assessed for PLAP expression (placental alkaline phosphatase) (60 kDa), a known biomarker of material of placental origin.

Flow Cytometric Analysis of STBEVs Derived from Placenta Perfusion Confirmed Co-Expression of DPPIV and PLAP

While Western blotting analysis measured the total DPPIV expression, flow cytometry was used to measure the expression of DPPIV on the surface of the larger STB-MVs. STB-EXs are not interrogateable by flow cytometry because the vesicles are below 300 nm (the lower limit of detection). Three colour flow cytometric data revealed that 41.07±8.10% of the STB-MVs population was PLAP and DPPIV positive while using a corresponding FMO control (FIG. 10). It was noted that 57.37±8.21% was solely labelled with PLAP. These results, together with the immunodepletion data, suggest that the majority of DPPIV in the STB-MVs is expressed together with PLAP.

Confirmation that STBEVs Co-Express DPPIV and PLAP by Immunodepletion

In order to demonstrate that DPPIV and PLAP were co-expressed on the same STB-EVs, immunodepletion using magnetic Dynabeads was used. STB-EVs were incubated with anti-DPPIV magnetic beads and vesicles pulled out by these beads were interrogated for DPPIV and PLAP by Western blotting. Both STB-MVs and STB-EXs showed DPPIV and PLAP positivity (FIG. 11 and FIG. 12). Confirmation of co-localizatin on the same vesicles was obtained by performing the converse experiment, namely, STBEVs pulled out with anti-PLAP magnetic beads expressed both PLAP and DPPIV. However, in the STB-MVs pool the PLAP pull out yielded a greater signal for DPPIV compared to the DPPIV pull out, which may indicate that some DPPIV is intravesicularly located. To assess the relative quantities of STB-EVs bound to DPPIV or PLAP, NTA analysis was used. NTA analysis for STB-MVs pool supernatants from the bead depletion experiments showed that 36.3% of STB-MVs were depleted with anti-DPPIV beads whilst 64.2% were positive for PLAP (FIG. 12). STB-EX pool supernatants demonstrated 8.63% DPPIV positivity and 31.78% PLAP positivity (FIG. 11). Analysis of the supernatant of IgG1 or IgG2a coated beads showed negligible depletion therefore excluding non-specific binding (FIG. 11-12).

DPPIV Activity

Having detected the presence of DPPIV in STBEVs samples, its activity in normal and GDM pregnancies was investigated. The results show enzyme activity was elevated in both STB-MVs (FIG. 14) and STB-EXs (FIG. 15) from women with GDM compared to normal subjects. A total of 12 women were included in the study. STB-MVs showed significantly higher DPPIV activity (p<0.05), while in STB-EXs the trend same was observed.

It was also demonstrated that it is possible to inhibit DPPIV present on the surface of STB-EVs isolated from placenta perfusion with a DPPIV specific inhibitor—Vildagliptin™ Both exosomes and microvesicles showed dose dependent inhibition with the addition of Vildagliptin™ (FIG. 13).

STBEVs in Uterine Vs Peripheral Plasma

Four colour flow cytometry was used to investigate the circulating levels of placental derived EVs in uterine and peripheral plasma (n=6). Syncytiotrophoblast microvesicles carrying DPPIV are present in significantly higher amounts in the uterine vein (DPPIV/PLAP double positive events per mL of plasma) compared with matched peripheral vein plasma (p<0.05), confirming the contribution of the placenta to the circulating levels of DPPIV during pregnancy (FIG. 16). The number of PLAP positive events was also significantly higher in uterine vein compared to peripheral plasma, again confirming the placental origin of circulating particles.

STBEVs in GDM Vs Normal Plasma

In this study for the first time, placental derived EVs carrying DPPIV are identified in peripheral plasma. The levels of those particles in the peripheral circulation of woman with normal and GDM pregnancies were investigated using flow cytometry. Significantly higher levels of STB-EVs expressing DPPIV were observed in GDM pregnancies compared to normal pregnancies. In GDM pregnancies from 2000 to 13200 PLAP/DPPIV positive events were observed in 1 mL of peripheral plasma, while in normal pregnancy only from 100 to 600 PLAP/DPPIV double positive events were observed.

Discussion

The data presented in this study indicates that DPPIV plays an important role in the pathophysiology of GDM. DPPIV is a glycoprotein that rapidly cleaves the N-terminal dipeptides of incretin hormones [such as glucagon like peptide (GLP-1)] that are known to increase insulin secretion and thus regulate glucose homeostasis. GLP-1 stimulates glucose-dependent insulin secretion, slows gastric emptying and increases β-cell mass. Circulating levels of GLP-1 declined significantly within just 2 minutes due to the degradation by the DPPIV. The findings subsequently led to the development of FDA approved class of drugs—DPPIV inhibitors. Therefore, we hypnotized that decreased insulin response in women with GDM is correlated with elevated levels of DPPIV during the gestation.

In data presented here shows that DPPIV is expressed on the syncytiotrophoblast layer, which constitutively secretes STB-EVs throughout pregnancy. It also confirms that DPPIV is co-expressed with placental alkaline phosphatase (marker of placental origin), as demonstrated using flow cytometry and immunobead depletion experiment.

The data shows that DPPIV activity is found to be significantly higher in GDM pregnancies than in normal controls, suggesting a physiological role for DPPIV in GDM. DPPIV activity from STB-EVs is shown to be inhibited by using FDA approved drugs—gliptins.

The use of a four-color Flow Cytometry enabled the presence of DPPIV on circulating placental derived microvesicles in both uterine vein and peripheral vein plasma of pregnant women to be observed. Syncytiotrophoblast microvesicles carrying DPPIV are present in significantly higher amounts in the uterine vein compared with matched peripheral vein plasma, confirming the contribution of the placenta to the circulating levels of DPPIV during pregnancy.

Finally, a significant difference was observed between GDM and normal pregnancies in with respect to the amount of DPPIV and PLAP double positive events observed in 100 μL of peripheral plasma. The results suggest that DPPIV is a good predictor of GDM, and could provide the basis of a therapeutic target and treatment opportunity for GDM, such as DPPIV inhibitors.

In conclusion, the results herein demonstrate for the first time the expression of DPPIV on STB-EVs. They also show that DPPIV is active and can be inhibited using FDA approved drugs. A difference in DPPIV expression and activity is also shown between normal and GDM pregnancies, suggesting it may have a role in the pathogenesis of gestational diseases with increased insulin resistance.

1. A method of determining the gestational diabetic status of a pregnant subject, comprising:
a) providing a biological sample obtained from the subject; and
b) determining the presence, and/or level, of syncytiotrophoblast extracellular vesicles in the biological sample.

2. The method of claim 1 further comprising determining the level of syncytiotrophoblast microvesicles, syncytiotrophoblast exosomes, or a combination thereof, in a sample from a subject.

3. The method of claim 1 or 2 further comprising determining the level of insulin receptor and/or dipeptidyl peptidase 4 (DPPIV) in the sample.

4. The method of any preceding claim wherein the level of either or both the insulin receptor and DPPIV in the sample is used to determine the level of syncytiotrophoblast extracellular vesicles in the sample.

5. The method of any preceding claim wherein an increase in the number of syncytiotrophoblast extracellular vesicles is diagnostic or prognostic for gestational diabetes.

6. The method of claim 5 wherein an increase in the number of syncytiotrophoblast microvesicles and/or the number of syncytiotrophoblast exosomes is diagnostic or prognostic for gestational diabetes.

7. A method of determining the gestational diabetic status of a pregnant subject, comprising:
a) providing a biological sample obtained from the subject; and
b) determining the level insulin receptor and/or DPPIV in the biological sample.

8. The method of claim 7 wherein an increase in the level of insulin receptor and/or DPPIV is diagnostic or prognostic for gestational diabetes.

9. The method of any preceding claim further comprising the step of measuring the level of circulating insulin in a subject.

10. The method of any preceding claim wherein the method further comprises the step of comparing the level determined in (b) with one or more reference values.

11. The method of claim 10 wherein the reference value may be the level of syncytiotrophoblast extracellular vesicles, and/or the level of insulin receptor and/or DPPIV, in a sample obtained i) from a pregnant subject who does not have/does not go on to develop gestational diabetes, or ii) a previous level observed in the subject at an earlier time period.

12. A method according to any preceding claim wherein the biological sample is blood, serum or plasma.

13. A method of treating gestational diabetes in a pregnant subject, comprising:
(a) obtaining a biological sample from the subject;
(b) determining the level of syncytiotrophoblast extracellular vesicles, and/or the level of insulin receptor and/or DPPIV, in the biological sample; and
(c) administering anti-diabetic therapy to the subject if the level of syncytiotrophoblast extracellular vesicles, and/or the level of insulin receptor and/or DPPIV, in the sample is indicative and/or diagnostic and/or predictive of gestational diabetes.

14. A method of diagnosing and treating gestational diabetes in a pregnant subject, comprising:
(a) obtaining a biological sample from the subject;
(b) determining the level of syncytiotrophoblast extracellular vesicles, and/or the level of insulin receptor and/or DPPIV, in the biological sample;
(c) diagnosing gestational diabetes when the level of syncytiotrophoblast extracellular vesicles, and/or the level of insulin receptor and/or the level of DPPIV is elevated; and
(d) administering an effective amount of an anti-diabetic therapy to the subject.

15. The method of claim 13 or 14 wherein the anti-diabetic therapy comprises one or more of insulin, metformin, an insulin receptor blocker, dialysis to remove syncytiotrophoblast vesicles, plasmapheresis, a DDPIV inhibitor, a gliptin or a combination thereof.

16. A kit for use in determining the gestational status of a pregnant subject comprising at least one agent for determining the level of syncytiotrophoblast extracellular vesicles, and/or the level of insulin receptor and/or DPPIV, in a biological sample obtained from the subject.

17. Use of the determination of the level of syncytiotrophoblast extracellular vesicles, and/or the level of insulin receptor and/or DPPIV, in a biological sample as a means of assessing the gestational diabetic status in a pregnant subject.

18. A method of determining the risk of a woman developing Type 2 diabetes post-partum, the method comprising determining the level of insulin receptor and/or DPPIV positive syncytiotrophoblast extracellular vesicles in a blood sample obtained from the woman at delivery.
